(19) United States
(12) Plant Patent Application Publication
(10) Pub. No.: US 2020/0337190 P1
(43) Pub. Date: Oct. 22, 2020

(54) CHESTNUT PLANT NAMED 'OZARK CHINQUAPIN-R'

(50) ;

(71) Applicant: Stephen H. Bost, Poplar Bluff, MO (US)

(72) Inventor: Stephen H. Bost, Poplar Bluff, MO (US)

(21) Appl. No.: 16/873,460

(22) Filed: Apr. 16, 2020

Related U.S. Application Data
(60) Provisional application No. 62/836,082, filed on Apr. 19, 2019.

(51) Int. Cl. A01H 6/00

(52) U.S. Cl. PLT/152

(57) ABSTRACT

Ozark Chinquapin-R is a new and distinct North American Castanea cultivar that is blight-resistant, precocious and a producer of heavy annual crops of one to six nuts per bur. It produces a heavy crop of nuts each year with a small-sized nut containing the highest protein and carbohydrate percent of any chestnut. Nut drop begins around September 10 and continues for a seven-to eight-week period. Most of the nuts produced drop in the first five weeks of the period of nut drop from this cultivar. Ozark Chinquapin-R is a medium-sized tree that has an elliptic-shaped canopy with high upright to spreading and diffuse lateral branches. This nine-year-old cultivar is 7.92 meters tall with a canopy covering an area 21.22 square meters. Its heavy nut crop occurs from early September through mid-November, providing the highest protein and carbohydrate nut crop of any chestnut for people and wildlife.

Latin name: Castanea ozarkensis.

Varietal denomination: Ozark Chinquapin-R.

### CROSS-REFERENCE TO RELATED APPLICATION

This application claims priority back to U.S. patent application Ser. No. 62/836,082 filed on Apr. 19, 2019, the contents of which are incorporated herein by reference.

### BACKGROUND OF THE INVENTION

The genus Castanea includes the Ozark chinquapin, the American chestnut and the Allegheny chinkapin. Over three billion trees from the genus Castanea have been lost in North American due to the chestnut blight Cryphonectria parasitica. The blight was accidentally imported on chestnut trees from Asia and was first noticed in New York City in 1904. The blight spread quickly killing trees by the fungus entering the bark and killing the cambium of the tree, cutting off vital nutrients. By the 1940s the chestnut blight Cryphonectria parasitica had reached the west side of the Mississippi River and began killing millions of Ozark chinquapin trees.

Before the arrival of the chestnut blight, the Ozark chinquapin was an abundant forest tree that provided a dependable high-protein nut crop every year without fail, and this crop was a very important food source for people and wildlife. The loss of this tree also had a negative impact on forest ecosystems that depended on it. The blight kill was so devastating that it was thought that no Ozark chinquapin tree had survived.

The object of the present invention is to provide an Ozark chinquapin tree that is resistant to the chestnut blight, has a high-yield nut crop and is fast-growing. This newly created cultivar is a dependable high-protein food source and an economic resource for people. This new cultivar will be a dependable food source for wildlife that is not present in forest today. Forest ecosystems will also benefit from this blight-resistant cultivar.

The present invention is the result of over fourteen years of field and laboratory research. This work has included hundreds of emails and calls networking with farmers, thousands of miles driven and rigorous hiking in rough terrain on a mission to discover surviving trees that exhibited some levels of blight resistance. This search encompassed seven states, and in February 2006, the inventor discovered the first tree that had not died of the blight. Other discoveries followed, and the inventor began collecting genetic material for a breeding program to enhance this desired trait. These trees were carefully selected from thousands of trees found in Missouri, Arkansas, Louisiana, Oklahoma, Texas, Mississippi and Alabama.

In the fall of 2006, remote locations for research test plots were established in Carter, Shannon, Dent and Cape Girardeau counties in Mo. These sites were selected because they had well-drained soils, were in secure locations to minimize vandalism, and had diverse plant populations and wildlife in immediate proximity. These requirements needed to be met to assure that these trees could be established in similar historic locations with minimal care and thrive.

Another important consideration for the breeding program was monitoring how Ozark Chinquapin-R could coexist with new threats that were not an issue 80 years ago. Today, resistance to the chestnut blight is the greatest threat to Ozark Chinquapin-R survival; however, new diseases, pathogens and invasive insects and plants are also threatening agriculture and forest ecosystems. For example, Ink Disease Phytophthora cinnamomiis present in the soils, and invasive species of Gall Wasp and leaf-eating Asiatic Oak Weevils have wreaked havoc for the last two decades on recovery efforts of the American chestnut.

Beginning in 2007 genetic material from these carefully selected trees was planted on research test plots. Further asexual reproduction and controlled pollination were conducted to increase blight resistance and produce higher nut yields and faster growth. This process has resulted in a cultivar with traits not found in wild Ozark chinquapin trees; the present invention is resistant to the chestnut blight and will produce up to six nuts per bur, making it multiple times more productive than wild cultivars. With double production not uncommon with this new cultivar, is precocious, growing 15% to 45% faster than wild Ozark chinquapin trees without fertilizer or soil amendments.

### BRIEF SUMMARY OF THE INVENTION

The present invention relates to a new and distinct Ozark chinquapin cultivar that is blight-resistant and highly productive. This new cultivar produces large nut crops of one to six nuts per bur, has the highest percentage of protein and carbohydrates of any chestnut, and was created by using stem grafts and controlled pollination. This method is still being used today on research test plots in Carter, Shannon, Dent and Cape Girardeau counties in Mo.

### BRIEF DESCRIPTION OF THE PHOTOGRAPHS

FIG. 1 is a photograph of wild Ozark chinquapin with only one nut per bur that splits into two equal halves.

FIG. 2 is a photograph of Ozark Chinquapin-R with two to six nuts per bur with the bur splitting into four equal parts.

FIG. 3 is a close-up photograph of Ozark Chinquapin-R with two to six nuts per bur with the nuts displaying flat sides instead of round with burs splitting into four equal parts instead of two.

FIG. 4 is a photograph of a typical blighted wild Ozark chinquapin tree killed by the blight with blighted stump sprouts and the old tree trunk.

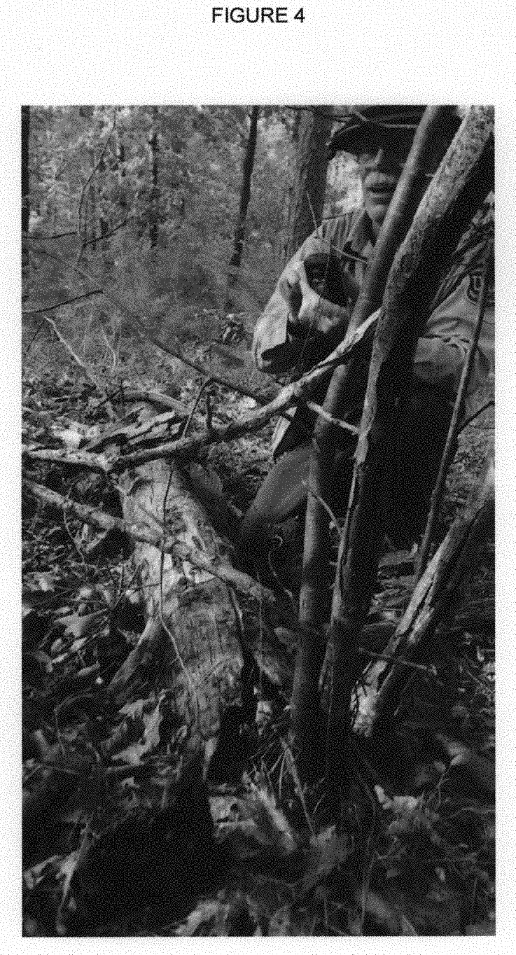

FIG. 5 is a photograph of one of the large blight-free Ozark chinquapin trees used to asexually create Ozark Chinquapin-R.

FIG. 6 is a photograph of wild Ozark chinquapin planted in 2009.

FIG. 7 is a photograph of the crossed tree used to produce Ozark Chinquapin-R planted in 2010 showing 15-45% more growth than wild Ozark chinquapin.

FIG. 8 is a photograph of a typical three to 15 nuts per branch in wild Ozark chinquapin.

FIG. 9 is a photograph of nut production on Ozark Chinquapin-R showing up to 47 nuts per branch.

FIG. 10 is a photograph of asexually reproducing Ozark chinquapin twigs grafted onto American chestnut conducted in 2006-2007.

FIG. 11 is a photograph of asexual reproduction using Ozark chinquapin scion wood grafting onto Chinese chestnut Castanea mollissima rootstock in 2018.

### DETAILED BOTANICAL DESCRIPTION

### A. Overview

The Ozark chinquapin, Castanea ozarkensis, is a species native to North America that will grow between 25° and 45° North latitudes. Labratory DNA studies indicate it has a more diverse gene pattern than the American chestnut or the Alleghany chinkapin and is the oldest Castanea in North America. The Ozark chinquapin is more resistant to chestnut blight Cryphonectria parasitica than the American chestnut Castanea dentata and less blight-resistant than the Chinese chestnut Castanea mollissima. Ozark chinquapins grow in soils that are low in organic material and are well-drained. The tap root of this tree serves as a survival mechanism in the first decade of life, allowing the tree to survive drought and extreme summer temperatures. These trees grow in acidic soils with PH 5.2-6.5. The name “chinquapin” is an Algonquin Indian word that translates to “little tasty chestnut.” Historic records from survey notes, newspaper articles, pollen analysis, recent discoveries of remnant populations and modern DNA testing verify that the Ozark chinquapin tree had a native range that extended from Texas and Oklahoma, through Georgia and into Virginia.

When the trees are young, the bark is smooth with a brown to gray color that resembles applewood. As the tree matures, the bark becomes long and deeply furrowed. Trees can grow to 24 meters tall and up to one-meter diameter at breast height (DBH) with deep saw-toothed leaves of a deep green with a silver cream color on the underside. In the fall, the leaves turn yellow before becoming brown and falling from the tree. On blighted stump sprouts, the dead leaves tend to cling to the branches through the winter. The leaves are often slightly wider in the middle; however, leaves on the same tree will have a narrower chestnut-like appearance. The pellicle or thin membranous skin on the nuts is thin and peels readily from the kernel. Trees are a spreading type in full sun with more of a vase shape as the tree matures, reaching a height of 25 meters. This is a medium age lived tree averaging 70-110 years. The trees will sometimes sprout one and sometimes two stems from the base of the tree. This phenomenon was also common in pre-blighted forest.

Ozark chinquapins are monoecious and cannot self-pollinate, requiring another pollinator. Staminate flowers appear on erect cylindrical catkins with 4-16 stamens. It is not uncommon on this new cultivar to produce nuts in the third year of growth. This new cultivar can produce catkins of staminate flowers the second year of growth. This cultivar can produce nuts the third year of growth by production of pistillate flowers. Nuts are small and chestnut brown in color. Ozark Chinquapin-R produces not just one nut per bur but typically two to six nuts per bur. Approximately 20% of burs produce multiple nuts and approximately 80% of burs produce a single nut. The burs that produce multiple nuts are approximately 25% larger than burs produce a single nut.

Ozark chinquapin-R is a medium-sized tree that is broadly elliptic in shape with high, upright and horizonal spreading branches. The Ozark Chinquapin-R tree is a nine-year-old tree 7.92 meters tall with a trunk DBH of 19.5 cm. The canopy is 5.18 meters wide with a canopy area of 21.22 sq. meters and potential maturity height of 0.3 to-1 meter.

Ozark Chinquapin-R produces a large nut crop every year. The nuts start dropping about September 10 and continue to drop for a seven-to eight-week period. A high percentage of the nuts produced by this cultivar drop during the first five weeks of the nuts' drop. Ozark Chinquapin-R is a medium sized tree that is blight-resistant, precocious, and produced one to six nuts per bur. These nuts are extremely high in protein and carbohydrates. The tree fruits annually and produces a large nut crop for the tree size. It also drops the nuts over an extended time period.

In 2018 at research test plots in Carter County, Mo., one 7.8 meters tall Ozark Chinquapin-R was checked for nut production in 2018. Carefully counting nuts collected each Tuesday for seven weeks yielded a total of 3,844 nuts collected. This yield is over twice the production of a same size wild Ozark chinquapin; however, trail camera pictures and videos indicated many more nuts than the 3,844 were produced but eaten by wildlife, including deer, turkeys, raccoons, squirrels, chipmunks, packrats, mice, voles, blue jays, woodpeckers and shrews, before they could be collected and counted. The true number produce is estimated to be approximately 6,000 nuts. This new cultivar could become an important food source for people, wildlife, and domestic livestock and may be used in agroforestry, wildlife food plots and orchards.

In 2006 the inventor discovered a rare Ozark chinquapin tree along the edge of a cultivated farm field in southwest Arkansas that exhibited levels of blight resistance and was used to help create Ozark Chinquapin-R. During the search for surviving trees that might have blight resistance, more than 2,700 Ozark chinquapin sprouts, stump sprouts, saplings, hybrids, Alleghany chinkapin, American chestnut and look-alike trees were observed in the field before the inventor carefully selected Ozark chinquapin trees that exhibited levels of blight resistance. Laboratory DNA tests were performed starting in 2007 to confirm that the trees being used in the breeding program were pure Ozark chinquapin trees that were not hybridized with any foreign or domestic Castanea. From this group of selected Ozark chinquapin trees that exhibited levels of blight resistance the number was narrowed down further by removing trees that exhibited undesirable traits such as low nut production, smaller than average size of nuts, slower growth rate and later sexual maturity. Repeated asexual reproduction and controlled pollination yielded desirable traits that were consistent and present in F1 generations. Continued field observations and laboratory blight testing confirm F2 generations were also consistent with the same desirable traits.

In February 2007 the inventor nut grafted stems from the first tree he discovered in southwest Arkansas bordering the cultivated farm field, collecting pollen from these clones the same year. The pollen was dried and frozen at −18C to be used later. In 2006 along a roadway in rural southwest Missouri the inventor discovered a unique Ozark chinquapin growing along a rural road by an orchard and large garden. The tree, unlike others he had discovered, had a large branch cut away from it by the rural electric coop and was not reacting to the blight as other trees the inventor had observed. Like the first tree found in southwest Arkansas, this tree exhibited levels of resistance to the blight. The inventor returned in late May 2007 and carefully pollinated the tree with the landowner's permission. The pollen used was collected from nut-grafts done with the tree from southwest Arkansas. He collected approximately 20 seeds from this controlled cross. In the spring of 2008 these crosses were planted on research test plots that were previously established. In 2010 the inventor collected pollen from these trees that demonstrated desired traits. Pollen from these select trees was then crossed with yet another controlled cross that demonstrated fast, vigorous growth, produced pollen in the second year and exhibited no indications of blight. Clones made with grafts from these trees and seed planted from them were carefully observed for the next three seasons. Careful controlled pollination of these trees continued, and more grafts were created to check the consistency of F1 and F2 generations. Desirable traits pertaining to high yield, multiple nuts per bur, and rapid growth were observed in the field, and laboratory tests confirmed high consistent blight resistance in F1 and F2 generations. Cloning with stem grafts, controlled pollination, planting seed from the cultivar and testing continue today for monitoring of desirable traits.

Tables 1 and 2 below illustrate the specific differences between the new cultivar Ozark Chinquapin-R and natural occurring wild populations of Ozark chinquapin. The present descriptions pertain to the variety as grown on research test plots in Carter, Shannon, Dent and Cape Girardeau counties in Mo.

TABLE 1
Characteristics New Variety

Tree:
Size-small-7.92 meters tall with a canopy width of 5.18 meters and a canopy
area of 21.22 sq. meters, potential maturity mature height of .3-1 meter.
Tree shape is broadly elliptic becoming vase like as it matures.
Vigor-vigorous
Trunk:
Form-upright with branches high and diffuse as well as extended lateral spread,
subbasicaulous common.
Texture-relatively smooth when young becoming deep furrowed in long strips
as it matures.
Color of bark-Brown or gray, becoming grayer as it matures.
Branches:
Form-upright to spreading
Texture-relatively smooth
Lenticels-few, small
Branching habit-horizonal spreading as well as lateral high and diffuse.
Color-new wood grey-brown, with grey as it matures.
Foliage:
Quantity-abundant
Density-dense
Leaves:
Size-varies from 16-27 cm
Width-varies from 5-8 cm
Leaf ratio-1.7-2.2
Shape-elliptical
Leaf tip-acute
Leaf base-narrowly rounded or wedge shaped, sometimes subobtuse.
Thickness-thin, leaf venation pinnate, moderately prominent abaxially
Texture-coriaceous
Margin-deeply serrated saw-toothed
Petiole-length 1.0 cm-1.4 cm
Petiole pubescence-glabrous to sparsely simple hairs
Color-adaxial surface deep green low luster, glabrous blade, abaxial silvery cream
surface, under magnification moderately to densely stellate hairs on blade.
Bloom:
Amount of bloom-heavy
Color-green changing to yellow then cream during 2-3 days of pollination.
Blooming period-late May to early June.
Age at which tree starts flowering-early, first year for grafts, 2-3 years for planted nuts.
Male flower-catkin length 10-18 cm
Male flower-stamen number per catkin 4-16
Female flower-flower number per bur 1-6
Female flower-style number per flower 4-7
Crop:
Bearing-regular annual bearer
Productivity-prolific
Ripening period-long, nuts begins dropping approx. September 10 and
continue for a 7-8-week period.
Distribution of nuts on tree-well distributed, fruits 16-28 cm from the terminals
with 6-47 burs.
Tenacity-burs crack while on tree and nuts release and drop from bur.
Hull:
Description-spiny round bur, splits into two halves if one nut,
splits into four equal parts if 2-6 nuts occur.
Size-3.8-4.2 cm in diameter for single nut, 3.5-5.5 cm for burs with 2-6 nuts
Number of nuts per bur-1-6 per bur
Dehiscence-splits easily when still on tree
Color-yellow-green-tan at dehiscence
Nut:
Size-small
Average size-1.55 cm × 1.72 cm for single round nut,
0.85 cm × 0.87 cm × 1.72 cm for 2-6 nuts per bur
Average weight-0.8 grams per nut, 567 nuts per pound.
Form-round, and others flattened on 2 side with 3-6 nuts, occ. 2,
hemispheric to somewhat ovoid on other side
Blossom end-broadly rounded with an obtuse tip
Basal end-broadly rounded to flat
Color-chestnut brown
Pubescence-glabrate
Shell-thin
Hardness of shell-relatively hard, yet not rigid
Texture of shell-smooth
Percentage of kernel to nut: very high +95%
Kernel:
Size-almost as large as nut size
Form-same as nut shape
Pellicle-thin, tan in color
Flavor-excellent, very sweet
Color-light-straw to tan
Resistance to insects:
Trees less than 3 meters tall experience leaf damage from Asiatic oak weevils
Cyrtepistomus castaneus.
Resistant to Oriental chestnut gall wasp Dryocosmus kuriphilus.
Resistance to disease:
Two laboratory tests confirm high resistance to chestnut blight Cryphonectria parasitica.
Resistant to Ink Disease Phytophthora cinnamomic.
Nut damage from Small Chestnut Weevils Curculio sayi.
Mammal damage:
Bark of trunks are damaged by the antlers of white tail deer, rabbits gnaw the
bark of young trees in winter, and black bears break limbs and damage
bark. Deer, bear, squirrels, voles, rabbits, mice, packrats, chipmunks
and variety of birds eat the nuts.

TABLE 2
Characteristics Wild Variety Ozark chinquapin

Tree:
Size-small tree or shrub like status, primary occurrence in form of
blighted stump sprouts-rarely reaching height great
than 5 meters because of cyclic chestnut blight kill.
Tree shape is shrubby to varies stages of blight killed stems.
Vigor-moderately vigorous
Trunk:
Form-dead stump visible in absence of fire, subbasicaulous blighted
stump sprouts, extremely rare to find tree form.
Texture-relatively smooth when young, rarely growing to becoming
deep furrowed in long strips as it matures.
Color of bark-brown or gray, becoming grayer as it matures.
Branches:
Form-upright to spreading
Texture-relatively smooth
Lenticels-few, small
Branching habit-horizonal spreading as well as lateral high and diffuse
Color-new wood grey-brown, with grey as it matures.
Foliage:
Quantity-minimal
Density-minimal
Leaves:
Size-varies from 16-27 cm
Width-varies from 5-8 cm
Leaf ratio-.6-1.1
Shape-elliptical in closed canopy, spreading in open canopy
Leaf tip-acute
Leaf base-narrowly rounded or wedge-shaped sometimes subobtuse
Thickness-thin, leaf venation pinnate, moderately prominent abaxially
Texture-coriaceous
Margin-12 to 25 deeply serrated saw-toothed
Petiole-length 1.0 cm-1.4 cm
Petiole pubescence-glabrous to sparsely simple hairs
Color-adaxial surface deep green low luster, glabrous blade,
abaxial silvery cream surface, under magnification
moderately to densely stellate hairs on blade.
Bloom:
Amount of bloom-minimal or nonexistent in closed canopy,
minimal to moderately heavy in open canopy.
Color-changing to yellow then cream during 2-3 days of pollination.
Blooming period-late May to early June.
Age at which tree starts flowering-nonexistent to 4-7 years.
Male flower-catkin length 10-18 cm
Male flower-stamen number per catkin 4-16
Female flower-flower number per bur 1
Female flower-style number per flower 4-7
Crop:
Bearing-historically an annual bearer, today almost nonexistent
Productivity-nonexistent to rare, monoecious needing another
pollinator, wildlife predation further limits viable seed base.
Ripening period-nonexistent to sporadic, nuts begins dropping approx.
September 10 to October 15.
Distribution of nuts on tree-nonexistent to sparse, if present usually
on upper branches.
Tenacity-burs crack while on tree and nuts release and drop from bur.
Hull:
Description-spiny round bur.
Size-3.8-4.2 cm in diameter for single nut.
Number of nuts per bur-1 nut per bur
Dehiscence-splits easily when still on tree
Color-yellow-green-tan at dehiscence, non-viable burs on tree
prematurely turn brown.
Nut:
Size-small.
Average size-1.55 cm × 1.72 cm for single round nut.
Average weight-0.8 grams per nut, 567 nuts per pound.
Form-round
Blossom end-broadly rounded with an obtuse tip
Basal end-broadly rounded to flat
Color-chestnut brown
Pubescence-glabrate
Shell-thin
Hardness of shell-relatively hard, yet not rigid
Texture of shell-smooth
Percentage of kernel to nut-very high +95%
Kernel:
Size-almost as large as nut size
Form-same as nut shape
Pellicle-thin, tan color
Flavor-excellent, very sweet
Color-light-straw to tan
Resistance to insects:
Trees less than 3 meters tall, leaf defoliation high from
Asiatic oak weevils Cyrtepistomus castaneus.
Resistant to Oriental chestnut gall wasp Dryocosmus kuriphilus.
Resistance to disease:
Chestnut blight Cryphonectria parasitica, has decimated
populations to the point tree has an S2 state rank in
Missouri as critically imperiled species of concern.
Resistant to Ink Disease Phytophthora cinnamomic.
Nut damage from Small Chestnut Weevils Curculio sayi.
Mammal damage:
Bark of trunks are damaged by the antlers of white tail deer,
rabbits gnaw the bark of young trees in winter, and black
bears break limbs and damage bark. Deer, bear, squirrels, voles,
rabbits, mice, packrats chipmunks and variety of birds eat nuts.

### B. Detailed Description of the Figures

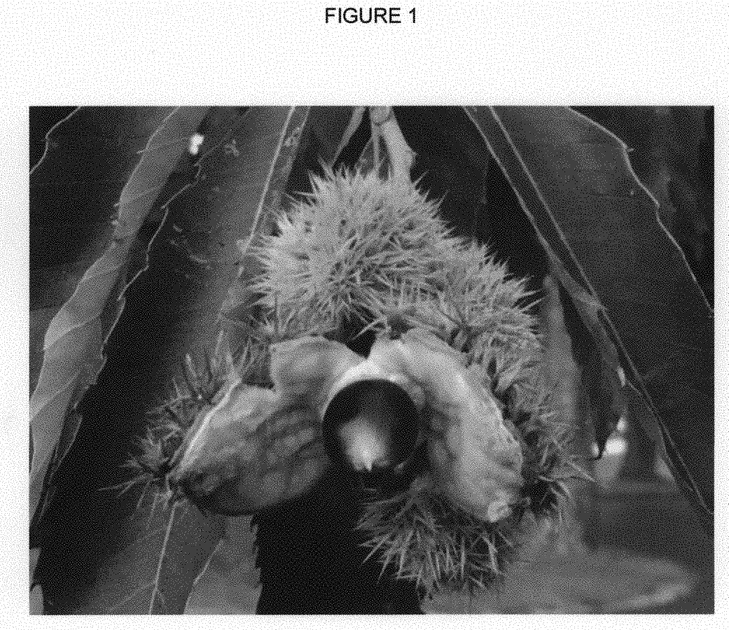

FIG. 1 is a photo of an Ozark chinquapin one would observe in the wild, with only one nut per spiky bur and the bur splitting into two equal halves as the nut ripens. The term chinquapin or chinkapin refers to a tree or bush that produces only one round nut per bur with the bur splitting into two equal halves as the nut ripens.

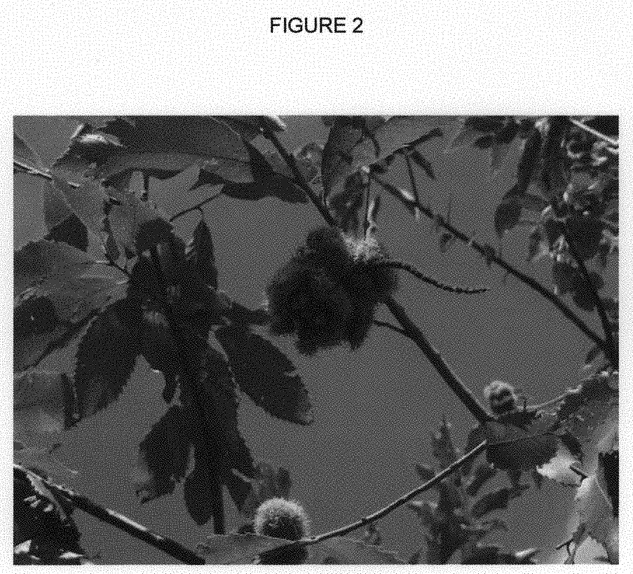

FIG. 2 is an example of Ozark Chinquapin-R producing multiple two to six nuts per spiky bur with the bur splitting into four equal parts as the nuts ripen. This unique ability to produce both multiple nuts per bur and burs with a single nut per bur is not found with wild cultivars. This new cultivar's ability to produce multiple nuts per bur occurs approximately 20% of the time, and this ability is steady and consistent with F1 and F2 generations. This unique ability to produce more than one nut per bur increases the annual nut yield of this new cultivar.

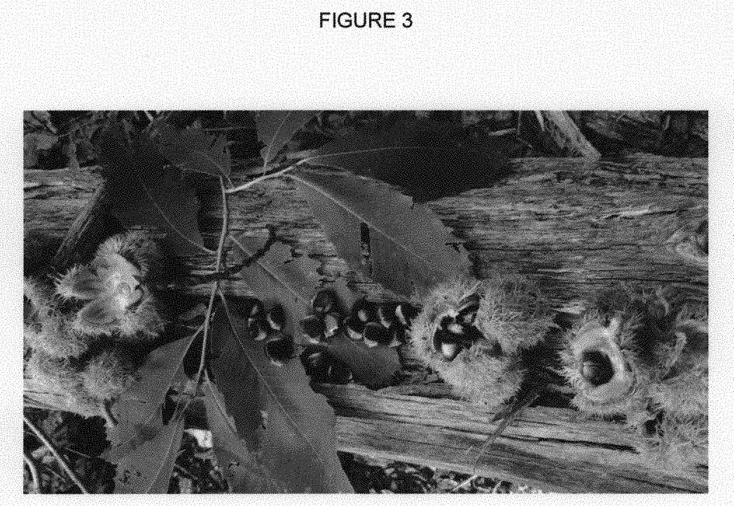

FIG. 3 provides an up-close view of Ozark Chinquapin-R with two to six nuts per spiky bur and the nuts displaying flat sides instead of the typical round nut of wild cultivars. The size of the burs with two to six nuts is approximately 25% larger than the single-nut burs the tree produces. Also visible are the spiky burs splitting into four equal parts instead of the two equal splits found on wild Ozark chinquapin trees.

This unique ability to produce not just one nut per bur but also two to six nuts per bur increases this new cultivar's productivity as compared to wild trees. Some of the smaller multiple nuts produced are easily eaten by smaller species of birds that could not normally swallow the larger sized Ozark chinquapin nut. This is significant because the smaller nuts produced by this cultivar can be a food source for smaller birds such as quail, ruffed grouse, woodpeckers and blue jays. Typically, only larger birds such as wild turkeys and crows can eat wild Ozark chinquapin nuts.

The larger nuts produced by American, European and Chinese chestnut are too large for smaller birds and mammals to eat. Ozark Chinquapin-R produces nuts that are smaller but have a superior flavor, and the smaller nut size is made up for by their concentrated amounts of protein, carbohydrates, unsaturated fats and trace minerals such as magnesium. Ozark Chinquapin-R nuts have double the protein content of the American chestnut and three to four times more protein than common species of red oak and white oaks. The nuts are the most sought-after food by wildlife in the forest. See Table 3 below.

TABLE 3¹⁻³
Protein, Carb., Crude Fat, Pot., Calcium, and Mag. Analysis

| Mast Species | Protein | Carb. | Fat. | Pot. | Cal. | Mag. |
|---|---|---|---|---|---|---|
| Ozark Chinquapin-R | 15.2% | 61.07% | 13.93% | .77% | .15% | .18% |
| Castanea Ozarkensis | | | | | | |
| White Oak | 4.6% | 46.60% | 2.90% | .08% | .22% | .05% |
| Quercus Alba | | | | | | |
| Scarlet Oak | 4.2% | 35.60% | 14.60% | .07% | .18% | .07% |
| Q. Cocinea | | | | | | |
| American Chestnut | 5-8% | 43.00% | 3-5% | n/a | n/a | n/a |
| C. Dentata | | | | | | |

As noted above, the nuts produced by Ozark Chinquapin-R are superior in percentage of carbohydrates and proteins compared to all chestnuts. See Table 4 below.

Visible in this photo (FIG. 3) is the now dead trunk of this once heathy wild Ozark chinquapin tree that was killed by the chestnut blight. The microbes in the soil kill the blight fungus so that the fungus only kills above the soil line. For this reason, sometimes (as is visible in this photo) two stems emerge from the still healthy unblighted root system only to die from the blight. In response to the die back of the tree trunk, the roots send up aggressive growing sprouts only to be killed every four to six years by the chestnut blight. The cycle of re-sprout and die back from the blight is typical of remnant populations of wild Ozark chinquapin trees found today. The third sprout that was healthy in the photo will eventually die as well.

In the wild it is extremely rare for an Ozark chinquapin tree to attain a height more than 15 feet before it is killed by the chestnut blight. Nut production is almost nonexistent because the tree's ability to reach the upper canopy of the forest and mature has been stopped by the blight. This has left the tree without a seed base to regenerate and rendered the tree functionally extinct.

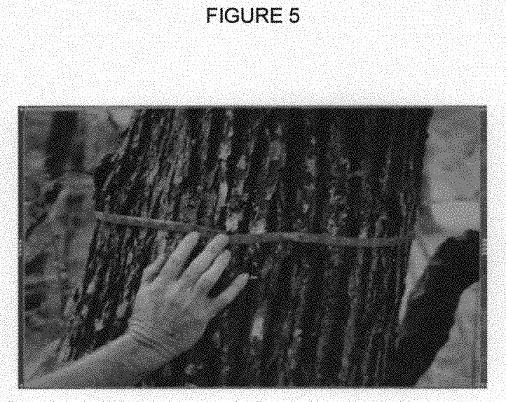

FIG. 5 is a photo of one of the rare Ozark chinquapin trees discovered along the edge of a cultivated farm field that exhibited levels of blight resistance and were used to help create Ozark Chinquapin-R. Wild Ozark chinquapin trees typically become blighted before they attain tree size and exist in the wild in shrub-like instead of as a tree.

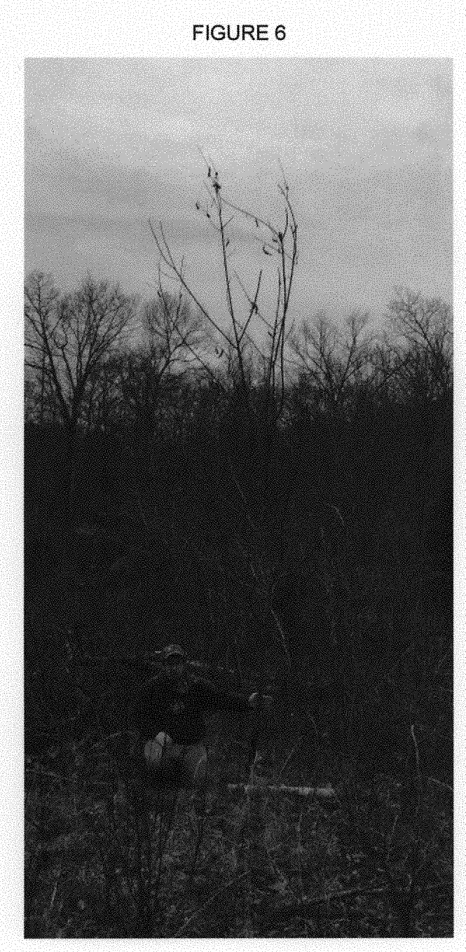

FIG. 6 is a photo that was taken on Feb. 19, 2019 of a wild Ozark chinquapin tree planted in the spring of 2009. This ten-year-old tree is 5.7 meters tall with a diameter of 7.0 centimeters at one-meter height.

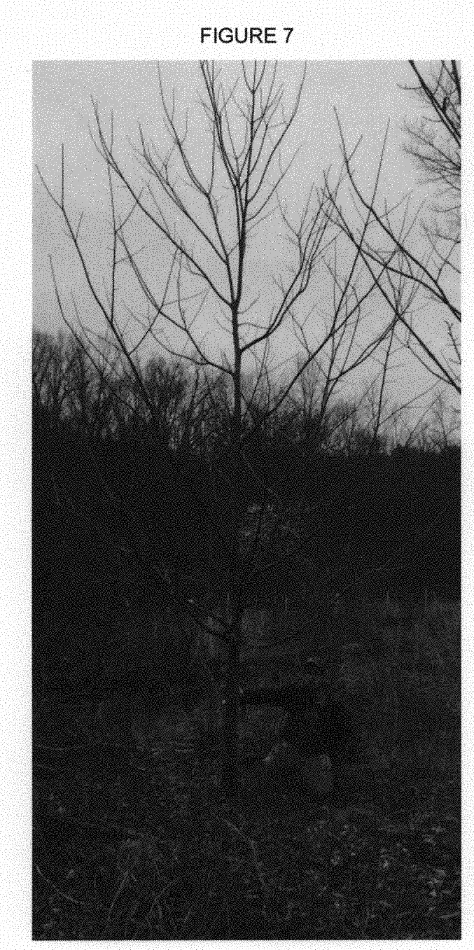

FIG. 7 is a photo that was also taken on Feb. 19, 2019 of an Ozark Chinquapin-R tree planted in spring of 2010. This nine-year-old Ozark Chinquapin-R was 7.8 meters tall with a diameter of 12 centimeters at one-meter height.

Both of the trees in FIGS. 6 and 7 were planted ten meters apart in full sun without the aid of fertilizers or any type of soil amendments or special treatment. Ozark Chinquapin-R was planted one year after the wild Ozark chinquapin and still achieved more growth.

Ozark Chinquapin-R is precocious with a growth rate 15-45% faster than a typical wild Ozark chinquapin. This rapid growth allows this new cultivar to produce nuts in three to five years as compared to four to seven years average with wild Ozark chinquapin trees. This rapid growth of Ozark Chinquapin-R is without aid of fertilizer or soil amendments.

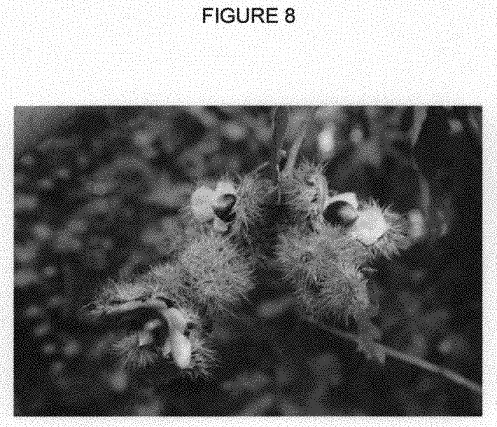

FIG. 8 is a rare photo of nut production on a wild Ozark chinquapin tree having three nuts ripening with their spiky burs opening near the end of a branch. If a wild tree manages to live long enough before being blight killed, it typically will produce three to 12 nuts near the end of a branch.

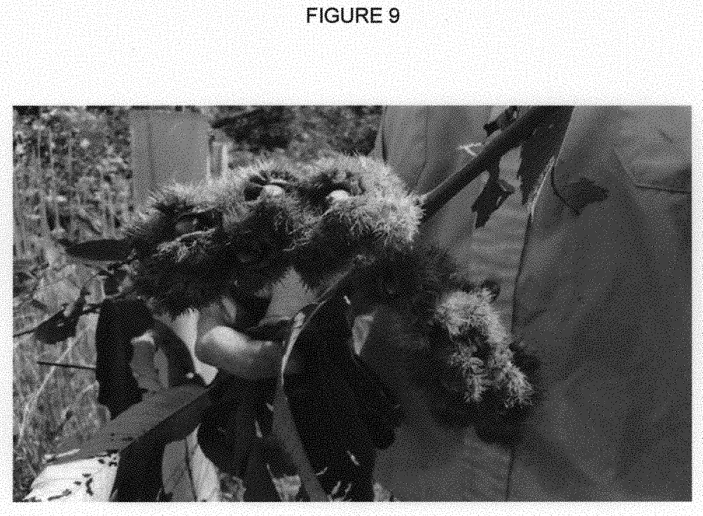

FIG. 9 is a photo showing the higher nut production on Ozark Chinquapin-R with up to 47 nuts per branch. Ozark Chinquapin-R has an extremely high nut crop yield.

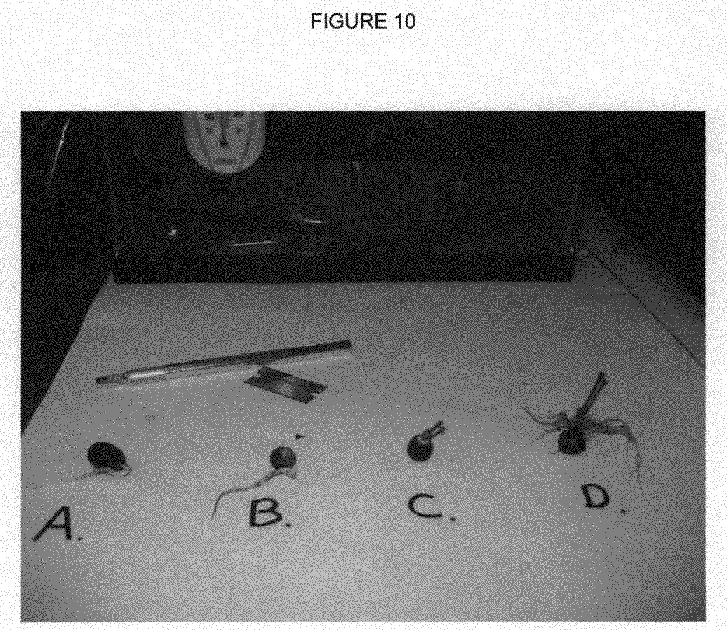

FIG. 10 is a photo of nut grafting first done in February 2007. This method of asexual reproduction allowed production of pollen from remote Ozark chinquapins trees that were hundreds of miles from research test plots. The selected trees from which scion wood was harvested for nut grafting exhibited levels of blight resistance but were difficult to access during pollination. Nut grafting produced clones that produced pollen the first year that was easy to obtain for strategic controlled pollination. Collected pollen was then dried and stored at minus 18 degrees Celcius to be used at a time months or years later.

FIG. 10 illustrates the process of nut grafting, namely:

1) The process begins with a germinated Ozark chinquapin nut, shown here with the root visible after being kept in cold storage for five months.

2) The tap root is cut away with razor blade, and the immature petiole is visible.

3) A dormant twig collected in February is removed from the tip of a mature branch of a selected Ozark chinquapin tree that will produce pollen. The twig is cut with an X-ACTO™ knife to a taped screwdriver-like bevel shape and carefully inserted into the immature petiole of the nut.

4) After three to four weeks of keeping the nut grafts at a continuous 18 to 24 degrees Celcius, the dormant twig comes to life, and the buds begin to swell. Later as if it were still on the mature (parent) tree, the twig leaves out and produces pollen.

This time-consuming method requires special attention to detail; watering with an eye dropper four times daily and carefully watering the root ends only so as not to allow any moisture to come into contact with the graft union. This method allowed pollen collection the first year from remote far away trees that were important for the breeding program to produce Ozark Chinquapin-R.

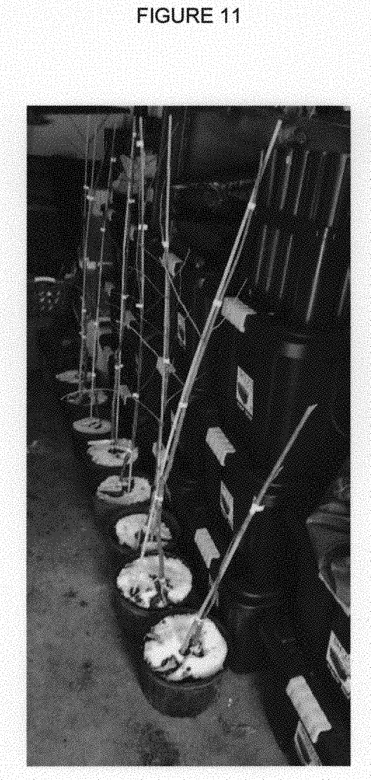

FIG. 11 is a photo of asexual reproduction using selected Ozark chinquapin and Ozark Chinquapin-R scion stems grafted onto root stock of Chinese chestnut Castanea mollissima. This asexual reproduction method was less time-consuming than nut grafting and accomplished three objectives: controlled pollination could be accomplished without use of lift bucket trucks in remote, roadless hard-to-reach tree locations; the Chinese root stock allowed them to grow in a greater variety of soils; and these grafts serve as a living genetic clone bank of selected trees for the breeding program to produce Ozark Chinquapin-R.

Ozark Chinquapin-R was created asexually along with controlled pollination from trees found in Missouri, Arkansas, Texas, Oklahoma, Louisiana, Mississippi and Alabama. The methods used to create this new cultivar include field and laboratory observations, plant morphology observations, DNA chloroplast tests, hypovirulence screening and testing, oxalic acid test, blight inoculation stem test, leaf assay blight testing, asexual reproduction using nut, bud and stem grafting, pollen collection, pollen processing, cold storage of pollen and controlled cross pollination. Other research methods performed with this new cultivar include hybridizing, genetically modifying, molecular research testing, clonal propagation, pollinating insects, medical research, micropropagation, use of soil microbes for blight control, air root pruning, inoculation of roots with root production method, building industry use, DNA research, medical use, tanning of animal skins, wood analysis, protein, fats, carbohydrate, cholesterol and trace mineral analysis, air root pruning, food industry use, orchards, agroforestry, livestock food, wood working, soil amendments and terra preta.

### REFERENCES

1. Bost, S. H. (2012). Ozark Chinquapin Nutritional Analysis SGS Laboratory, Unpublished Manuscript.

2. Bonner, F. T. (1974a). Seed Biology and Technology of Quercus. Res. Note SO-183. New Orleans, La.: U.S. Department of Agriculture, Forest Service, Southern Forest Experiment Station.

3. D. Fulbright, Personal Communication, February 2012.

I claim:

1. A new and distinct cultivar of the species Castanea named ‘Ozark Chinquapin-R’ as described and illustrated herein.
